(19) United States
(12) Patent Application Publication
(10) Pub. No.: US 2024/0027471 A1
(43) Pub. Date: Jan. 25, 2024

(54) MARKERS FOR PREDICTING CORONAVIRUS DISEASE 2019 (COVID-19) IMMUNE CHECKPOINT STORM, APPLICATION AND KIT THEREOF

(71) Applicant: Beijing Immupeutics Medicine Technology Limited, Daxing District Beijing (CN)

(72) Inventors: Ning SHEN, Daxing District Beijing (CN); Yaxian KONG, Daxing District Beijing (CN); Xiufang WANG, Daxing District Beijing (CN); Henghui ZHANG, Daxing District Beijing (CN); Jin SONG, Daxing District Beijing (CN)

(21) Appl. No.: 18/024,620

(22) PCT Filed: Jun. 22, 2021
(86) PCT No.: PCT/CN2021/101443

§ 371 Date: Mar. 3, 2023

(30) Foreign Application Priority Data
Sep. 3, 2020 (CN) 202010915265.X

(51) Int. Cl. G01N 33/68; G01N 33/543; G01N 33/58

(52) U.S. Cl. CPC G01N 33/6893; G01N 33/54313; G01N 33/58; G01N 2800/52

(57) ABSTRACT

Provided in the present application are markers for predicting Coronavirus disease 2019 (COVID-19) immune checkpoint storm, an application and a kit thereof, and a preparation method of the kit, belonging to the technical field of biomedicine. The markers of the present application comprise one or more of BTLA, GITR, HVEM, IDO, LAG-3, PD-1, PD-L1, TIM-3, CD28, CD80, 4-1BB, CD27, and CD152. Predicting the COVID-19 immune checkpoint storm by using the biomarkers provided by the present application or the kit prepared therefrom has the advantages of rapid and accurate detection at low cost, and has a broad prospect of application.

### TECHNICAL FIELD

The present application belongs to the technical field of biomedicine, and in particular to markers for predicting coronavirus disease 2019 (COVID-19) immune checkpoint storm and an application thereof, as well as a method for preparing a kit.

### BACKGROUND

As COVID-19 continues to spread worldwide, the related epidemic has become a global public health crisis. In addition to dyspnea, hypoxemia, acute respiratory distress, and cytokine release syndrome, progressive lymphopenia (especially T cells) is a prominent clinical feature of severe COVID-19. Recently, some studies have found that there is a correlation between T-cell deficiency and increased expression of several inhibitory checkpoint molecules on T cells in severe COVID-19 cases. The inhibitory checkpoint molecules have been demonstrated to be the critical factors for regulating T cell exhaustion in various chronic viral infections and tumor patients. It is further shown in recent studies that the inhibitory checkpoint molecules play a key role in the pathophysiology of acute viral infections (e.g., Ebola virus or Hantaan virus infection). Soluble isoforms of the inhibitory checkpoint can be generated by cleavage of membrane-bound proteins or alternative splicing of mRNA and competitively regulate the function of their membrane-bound proteins. Therefore, it is of great significance to develop soluble checkpoint molecular markers capable of predicting the immune imbalance of COVID-19 and differentiating the severity of patients.

Macrophages, neutrophil chemokines, pro-inflammatory cytokines, and anti-inflammatory cytokines are higher in the plasma of severe patients than in common influenza patients. After the human body is invaded by virus, the immune cells in the body release a large number of cytokines promptly, resulting in a “suicidal” effect and strengthened inflammatory response throughout the body, thus causing the progression of severe disease. In addition, in severe cases, the virus-specific T cells in the body are depleted after over-activation, and the level of immune response is reduced, thus reducing the antiviral capacity of the body. However, there are currently no biomarkers that can predict the occurrence of immune checkpoint storms in patients.

### SUMMARY

The present application aims to provide markers for predicting COVID-19 immune checkpoint storm, an application and a kit thereof, as well as a preparation method of the kit.

The present application provides markers for predicting COVID-19 immune checkpoint storm, wherein the markers include one or more of BTLA, GITR, HVEM, IDO, LAG-3, PD-1, PD-L1, TIM-3, CD28, CD80, 4-1BB, CD27, and CD152.

For the biomarkers of the present application, preferably, the COVID-19 includes 2019 novel coronavirus pneumonia and other organ damage diseases caused by 2019 Novel Coronavirus (2019-nCoV).

The present application further provides an application of the markers described in the above technical solution in the preparation of a kit for predicting COVID-19 immune checkpoint storm.

The present application further provides a kit for predicting COVID-19 immune checkpoint storm markers, wherein the kit includes: encoded microspheres coated with one or more capture antibodies of BTLA, GITR, HVEM, IDO, LAG-3, PD-1, PD-L1, TIM-3, CD28, CD80, 4-1BB, CD27, and CD152, respectively, one or more detection antibodies of BTLA, GITR, HVEM, IDO, LAG-3, PD-1, PD-L1, TIM-3, CD28, CD80, 4-1BB, CD27, and CD152 labeled respectively with a biotin, and streptavidin-labeled phycoerythrin.

The content of each component in the kit is not particularly limited in this application, and the ratio of the component content can be adjusted by those skilled in the art according to the actual situation of the test. Further preferably, the relationship between the amounts of the components in the kit of this application in the same system is as follows:
carboxyl microspheres: 0.4×10⁶ to 1.6×10⁶;
capture antibodies: each 30 to 70 μg of BTLA, GITR, HVEM, IDO, LAG-3, PD-1, PD-L1, TIM-3, CD28, CD80, 4-1BB, CD27, and CD152 capture antibodies;
detection antibodies: each 0.6 to 1.4 mg of BTLA, GITR, HVEM, IDO, LAG-3, PD-1, PD-L1, TIM-3, CD28, CD80, 4-1BB, CD27, and CD152 detection antibodies;
biotin: 0.6 to 1.4 mg;
streptavidin-labeled phycoerythrin: not particularly limited in this application, being conventional commercially available products in the art or being prepared by conventional methods known in the art, and its dosage can be determined by referring to the instructions of commercially available products or according to conventional means in the art and is not particularly limited herein.

Preferably, the clone numbers of the BTLA, GITR, HVEM, IDO, LAG-3, PD-1, PD-L1, TIM-3, CD28, CD80, 4-1BB, CD27, and CD152 capture antibodies are 6F7, 110416, LH1, 7H8L17, C9B7W, J116, MIH1, F38-2E2, 10F3, 18, 4B4, O323, 14D3, respectively.

Preferably, the clone numbers of the BTLA, GITR, HVEM, IDO, PD-L1, CD28, CD80, and CD152 detection antibodies are MIH26, DT5D3, eBioHVEM-122, 2E2.6, 10F.9G2, 37407, MEM-233, and WKH 203, respectively, and the LAG-3, PD-1, TIM-3, 4-1BB, CD27 detection antibodies are polyclonal antibodies.

Preferably, the encoded microspheres include carboxyl microspheres.

Preferably, the biotin includes N-carboxyl succinimide-activated biotin.

The present application further provides a method for preparing any one of the kits above, which includes the following steps:
preparation of encoded microspheres coated with capture antibodies: coupling one or more capture antibodies of BTLA, GITR, HVEM, IDO, LAG-3, PD-1, PD-L1, TIM-3, CD28, CD80, 4-1BB, CD27, and CD152 with corresponding encoded microspheres, respectively, to obtain encoded microspheres coated with one or more capture antibodies of BTLA, GITR, HVEM, IDO, LAG-3, PD-1, PD-L1, TIM-3, CD28, CD80, 4-1BB, CD27, and CD152, respectively;
preparation of biotin-labeled detection antibodies: ligating a biotin onto one or more detection antibodies of BTLA, GITR, HVEM, DO, LAG-3, PD-1, PD-L1, TIM-3, CD28, CD80, 4-1BB, CD27, and CD152, respectively, to obtain one or more detection antibodies of BTLA, GITR, HVEM, IDO, LAG-3, PD-1, PD-L1, TIM-3, CD28, CD80, 4-1BB, CD27, and CD152 respectively labeled with the biotin.

### Beneficial Effects

The present application provides serological biomarkers for predicting COVID-19 immune checkpoint storm, wherein the biomarkers are one or more of BTLA, GITR, HVEM, IDO, LAG-3, PD-1, PD-L1, TIM-3, CD28, CD80, 4-1BB, CD27, and CD152. It has been shown in the experiments that, for COVID-19 patients in severe or critical condition with a high risk of “immune checkpoint storm”, the baseline concentrations of BTLA, GITR, HVEM, IDO, LAG-3, PD-1, PD-L1, TIM-3, CD28, CD80, 4-1BB, CD27, and CD152 in their sera are all significantly higher than those in COVID-19 patients in mild or moderate condition and asymptomatic cases with a low risk of “immune checkpoint storm”.

Predicting the COVID-19 immune checkpoint storm by using one or more biomarkers of BTLA, GITR, HVEM, IDO, LAG-3, PD-1, PD-L1, TIM-3, CD28, CD80, 4-1BB, CD27, and CD152 provided by the present application or the kit prepared therefrom has the advantages of rapid and accurate detection at low cost, and has a broad prospect of application.

### BRIEF DESCRIPTION OF THE DRAWINGS

FIGS. 1-13 show the standard curve schematic diagrams of the biomarkers BTLA, GITR, HVEM, IDO, LAG-3, PD-1, PD-L1, TIM-3, CD28, CD80, 4-1BB, CD27, and CD152 in the test serum, in sequence;

FIG. 14 are diagrams respectively showing the difference in the concentrations of BTLA, GITR, HVEM, IDO, LAG-3, PD-1, PD-L1, TIM-3, CD28, CD80, 4-1BB, CD27, and CD152 in the sera of three groups of COVID-19 patients in asymptomatic, mild or moderate, and severe or critical condition, respectively, as detected by a liquid chip kit (the concentrations of cytokines in the severe or critically ill group are all higher than those in the mild or moderate group and the asymptomatic group);

FIG. 15 shows the receiver operating characteristic curves (ROC curves) for the prediction effectiveness of BTLA, GITR, HVEM, DO, LAG-3, PD-1, PD-L1, TIM-3, CD28, CD80, 4-1BB, CD27, and CD152 corresponding to the immune checkpoint storm in COVID-19 patients, respectively, with the area under the curve (AUC) and 95% confidence interval (95% CI) corresponding to each cytokine shown in the figure.

### DETAILED DESCRIPTION

The present application provides serological biomarkers for predicting COVID-19 immune checkpoint storm, wherein the serological biomarkers include one or more of BTLA, GITR, HVEM, IDO, LAG-3, PD-1, PD-L1, TIM-3, CD28, CD80, 4-1BB, CD27, and CD152. The present application has found through experiments that, the baseline concentrations of BTLA, GITR, HVEM, IDO, LAG-3, PD-1, PD-L1, TIM-3, CD28, CD80, 4-1BB, CD27, and CD152 in the sera of a patient are related to the occurrence of immune checkpoint storm. By using the baseline concentrations of BTLA, GITR, HVEM, IDO, LAG-3, PD-1, PD-L1, TIM-3, CD28, CD80, 4-1BB, CD27, and CD152 (the thresholds are 759.495 pg/mL, 15.265 pg/mL, 20.645 pg/mL, 79.540 pg/mL, 148.595 pg/mL, 42.465 pg/mL, 7.030 pg/mL, 999.280 pg/mL, 358.420 pg/mL, 158.780 pg/mL, 34.620 pg/mL, 470.330 pg/mL, 218.025 pg/mL, respectively) in the sera of the patient alone, the risk degree of immune checkpoint storm can be predicted. Therefore, the effect of predicting the COVID-19 immune checkpoint storm with BTLA, GITR, HVEM, IDO, LAG-3, PD-1, PD-L1, TIM-3, CD28, CD80, 4-1BB, CD27, and CD152 as the serological biomarkers has the advantages of high accuracy, convenient implementation, and low cost, and has a broad prospect of application.

The present application further provides an application of the serological biomarkers described in the above technical proposal in the preparation of a kit for predicting COVID-19 immune checkpoint storm. In the present application, the application includes any kits having the function of specifically detecting the above biomarkers prepared based on the above biomarkers.

The present application further provides a kit for predicting COVID-19 immune checkpoint storm, which includes: encoded microspheres respectively coated with BTLA, GITR, HVEM, IDO, LAG-3, PD-1, PD-L1, TIM-3, CD28, CD80, 4-1BB, CD27, and CD152 capture antibodies, BTLA, GITR, HVEM, IDO, LAG-3, PD-1, PD-L1, TIM-3, CD28, CD80, 4-1BB, CD27, and CD152 detection antibodies labeled respectively with a biotin, and streptavidin-labeled phycoerythrin. In the present application, the clone numbers of the BTLA, GITR, HVEM, IDO, LAG-3, PD-1, PD-L1, TIM-3, CD28, CD80, 4-1BB, CD27, and CD152 capture antibodies are preferably obtained or available to those skilled in the art from the prior art respectively, such as, 6F7, 110416, LH1, 7H8L17, C9B7W, J116, MIH1, F38-2E2, 10F3, 18, 4B4, O323, and 14D3. The clone numbers of the preferred BTLA, GITR, HVEM, IDO, PD-L1, CD28, CD80, and CD152 detection antibodies are MIH26, DT5D3, eBioHVEM-122, 2E2.6, 10F.9G2, 37407, MEM-233, and WKH 203, respectively, and the LAG-3, PD-1, TIM-3, 4-1BB, CD27 detection antibodies are polyclonal antibodies. In the present application, the encoded microspheres are preferably carboxyl microspheres. In the present application, the biotin is preferably N-carboxyl succinimide-activated biotin. In the present application, the kit firstly captures the BTLA, GITR, HVEM, IDO, LAG-3, PD-1, PD-L1, TIM-3, CD28, CD80, 4-1BB, CD27, and CD152 in the sample to test with the encoded microspheres respectively coated with the BTLA, GITR, HVEM, IDO, LAG-3, PD-1, PD-L1, TIM-3, CD28, CD80, 4-1BB, CD27, and CD152 capture antibodies, then quantifies the captured BTLA, GITR, HVEM, IDO, LAG-3, PD-1, PD-L1, TIM-3, CD28, CD80, 4-1BB, CD27, and CD152 respectively with the BTLA, GITR, HVEM, IDO, LAG-3, PD-1, PD-L1, TIM-3, CD28, CD80, 4-1BB, CD27, and CD152 detection antibodies labeled respectively with a biotin, and the streptavidin-labeled phycoerythrin. In the present application, the encoded microspheres means that microspheres with different fluorescent ratios are used for data encoding, the fluorescently encoded microspheres are covalently cross-linked with specific monoclonal antibodies, and individual microspheres are identified by laser scanning the fluorescent codes.

Based on the liquid chip technology, the present application develops a kit that can rapidly detect the BTLA, GITR, HVEM, IDO, LAG-3, PD-1, PD-L1, TIM-3, CD28, CD80, 4-1BB, CD27, and CD152 biomarkers in the serum, that is, the liquid chip kit. Such a kit has the advantages of no side effects, high sensitivity, rapid detection, good repeatability, etc. Moreover, the method for preparing such a liquid chip kit is simple and reliable, and has a good stability.

In a specific embodiment of the present application, the method for preparing the kit preferably includes the following steps:
(1) coupling capture antibodies coated with one or more of BTLA, GITR, HVEM, IDO, LAG-3, PD-1, PD-L1, TIM-3, CD28, CD80, 4-1BB, CD27, and CD152 respectively with encoded microspheres, to obtain encoded microspheres coated with one or more capture antibodies of BTLA, GITR, HVEM, IDO, LAG-3, PD-1, PD-L1, TIM-3, CD28, CD80, 4-1BB, CD27, and CD152;
(2) ligating a biotin onto one or more detection antibodies of BTLA, GITR, HVEM, IDO, LAG-3, PD-1, PD-L1, TIM-3, CD28, CD80, 4-1BB, CD27, and CD152, respectively, to obtain one or more detection antibodies of BTLA, GITR, HVEM, IDO, LAG-3, PD-1, PD-L1, TIM-3, CD28, CD80, 4-1BB, CD27, and CD152 respectively labeled with the biotin; there is no sequential relationship between the steps (1) and (2).

In the present application, one or more capture antibodies of BTLA, GITR, HVEM, IDO, LAG-3, PD-1, PD-L1, TIM-3, CD28, CD80, 4-1BB, CD27, and CD152 are coupled with encoded microspheres, to obtain encoded microspheres respectively coated with one or more capture antibodies of BTLA, GITR, HVEM, IDO, LAG-3, PD-1, PD-L1, TIM-3, CD28, CD80, 4-1BB, CD27, and CD152. In the present application, the coupling method preferably includes the following steps:
a. taking carboxyl microspheres, shaking the microsphere suspension by a vortex oscillator for 15 to 25 s to mix the microspheres evenly;
b. taking 0.4×10⁶ to 1.6×10⁶ carboxyl microspheres after shaking, transferring them into a centrifuge tube and centrifuging at ≥8000 g for 1.5 to 3 min to precipitate the microspheres;
c. removing the supernatant, adding 80 to 120 μL of dH₂O, shaking by a vortex oscillator for 15 to 25 s to resuspend the microspheres, centrifuging at ≥8000 g for 1.5 to 3 min to precipitate the carboxyl microspheres; removing the supernatant, adding 60 to 100 μL of 80 to 120 mmol/L sodium dihydrogen phosphate solution with a pH value of 6 to 6.5, and shaking by a vortex oscillator for 15 to 25 s to resuspend the washed carboxyl microspheres;
d. adding 8 to 12 μL of 40 to 60 mg/mL N-hydroxysulfosuccinimide, and shaking by the vortex oscillator gently;
e. adding 8 to 12 μL of 40 to 60 mg/mL 1-ethyl-3[3-(dimethylamino)propyl]carbodiimide, and shaking by the vortex oscillator gently;
f. incubating at room temperature for 15 to 25 min, shaking by the vortex oscillator gently every 8 to 12 min, centrifuging at ≥8000 g for 1.5 to 3 min to precipitate the activated carboxyl microspheres;
g. removing the supernatant, adding 40 to 60 mmol/L 2-(N-morpholino)ethanesulfonic acid (MES) with a pH value of 4.8 to 5.2, shaking by the vortex oscillator for 15 to 25 s to resuspend the activated carboxyl microspheres, centrifuging at ≥8000 g for 1.5 to 3 min to precipitate the washed carboxyl microspheres; repeating this step 2 to 3 times, washing 2 to 3 times with 40 to 60 mmol/L MES with a pH value of 4.8 to 5.2, adding 40 to 60 mmol/L MES with a pH value of 4.8 to 5.2, shaking by the vortex oscillator for 15 to 25 s, adding 30 to 70 μg of BTLA, GITR, HVEM, IDO, LAG-3, PD-1, PD-L1, TIM-3, CD28, CD80, 4-1BB, CD27, and CD152 capture antibodies, respectively into the well-mixed microspheres, metering to 400 to 600 μL with 40 to 60 mmol/L MES with a pH value of 4.8 to 5.2, and mixing evenly by the vortex oscillator; incubating at room temperature on a shaker for 1.5 to 3 h, and centrifuging at ≥8000 g for 1.5 to 3 min to precipitate the coupled microspheres;
h. removing the supernatant, adding 200 to 400 μL of PBS-TBN, shaking by the vortex oscillator for 25 to 35 s; incubating at room temperature on a shaker for 25 to 35 min, and centrifuging at ≥8000 g for 1.5 to 3 min to precipitate the coupled microspheres;
i. removing the supernatant, adding 0.8 to 1.2 mL of PBS-TBN, shaking by the vortex oscillator for 25 to 35 s, centrifuging at ≥8000 g for 1.5 to 3 min to precipitate the coupled microspheres; repeating this step 1 to 2 times, and washing 2 to 3 times with PBS-TBN;
j. adding 0.8 to 1.2 mL of PBS-TBN to resuspend the coupled and washed microspheres, obtaining the conjugates of BTLA, GITR, HVEM, IDO, LAG-3, PD-1, PD-L1, TIM-3, CD28, CD80, 4-1BB, CD27, and CD152 capture antibodies with the microspheres;
k. counting the number of the microspheres with a cell counter, with a concentration of 2 to 3×10⁵ per mL; and storing the coupled microspheres at 2 to 6° C. in dark.

In the present application, a biotin is ligated onto one or more detection antibodies of BTLA, GITR, HVEM, IDO, LAG-3, PD-1, PD-L1, TIM-3, CD28, CD80, 4-1BB, CD27, and CD152, to obtain one or more detection antibodies of BTLA, GITR, HVEM, LAG-3, PD-1, PD-L1, TIM-3, CD28, CD80, 4-1BB, CD27, and CD152 respectively labeled with the biotin. In the present application, the ligation method preferably includes the following steps:
\textcircled{1} diluting 0.6 to 1.4 mg of BTLA, GITR, HVEM, IDO, LAG-3, PD-1, PD-L1, TIM-3, CD28, CD80, 4-1BB, CD27, and CD152 detection antibodies respectively with 0.08 to 0.12 mol/L sodium bicarbonate buffer solution with a pH value of 7.8 to 8.2 to 0.6 to 1.4 mg/mL, with its final volume being 0.8 to 1.2 mL;
\textcircled{2} fully dialyzing the proteins alternately with 0.08 to 0.12 mol/L sodium bicarbonate buffer solution with a pH value of 7.8 to 8.2;
\textcircled{3} dissolving 0.6 to 1.4 mg of N-hydroxy succinimide-activated biotin with 0.8 to 1.2 mL of dimethyl sulfoxide;
\textcircled{4} adding 100 to 150 μL of 0.8 to 1.2 g/L NHSB solution respectively into 0.8 to 1.2 mL solution of BTLA, GITR, HVEM, IDO, LAG-3, PD-1, PD-L1, TIM-3, CD28, CD80, 4-1BB, CD27, and CD152 detection antibodies; continuously stirring at room temperature and keeping for 2 to 4 h

\textcircled{5} adding 9 to 10 μL of 0.8 to 1.2 mol/L NH₄Cl solution, stirring at room temperature for 8 to 12 min, fully dialyzing against PBS at 2 to 6° C. to remove the free biotin; screening 0.8 to 1.2 mL of molecules on the sample over a column, eluting slowly with PBS to collect 0.8 to 1.2 mL per tube, and eluting the proteins between 1 and 3 mL; the final concentrations of the added samples being 0.4 to 0.6 g/L of sodium azide and 0.8 to 1.2 g/L of BSA; and storing the conjugated products at 2 to 6° C. in dark.

The source of streptavidin-labeled phycoerythrin is not particularly limited in the present application, and it can be conventional commercially available products in the art or prepared by conventional methods known in the art.

In the present application, the markers are used in the method for judging the COVID-19 immune checkpoint storm, which preferably includes the following steps:
(1) measuring the contents of BTLA, GITR, HVEM, IDO, LAG-3, PD-1, PD-L1, TIM-3, CD28, CD80, 4-1BB, CD27, and CD152 markers in the serum sample of a subject;
(2) judging the occurrence risk of the COVID-19 immune checkpoint storm by using the measurements of BTLA, GITR, HVEM, IDO, LAG-3, PD-1, PD-L1, TIM-3, CD28, CD80, 4-1BB, CD27, and CD152 obtained in step (1).

In the present application, the COVID-19 includes 2019 novel coronavirus pneumonia and other organ damage diseases caused by 2019 Novel Coronavirus (2019-nCoV). In severe COVID-19 cases, there is a correlation between T-cell deficiency and increased expression of several inhibitory checkpoint molecules on T cells. The method provided in the present application can be applied to COVID-19 patients. For COVID-19 patients, the baseline concentrations of BTLA, GITR, HVEM, IDO, LAG-3, PD-1, PD-L1, TIM-3, CD28, CD80, 4-1BB, CD27, and CD152 in the sera of patients with a high risk of immune checkpoint storm are significantly higher than those in patients with a low risk. By using the baseline concentrations of BTLA, GITR, HVEM, IDO, LAG-3, PD-1, PD-L1, TIM-3, CD28, CD80, 4-1BB, CD27, and CD152 (the thresholds are 759.495 pg/mL, 15.265 pg/mL, 20.645 pg/mL, 79.540 pg/mL, 148.595 pg/mL, 42.465 pg/mL, 7.030 pg/mL, 999.280 pg/mL, 358.420 pg/mL, 158.780 pg/mL, 34.620 pg/mL, 470.330 pg/mL, 218.025 pg/mL, respectively) in the sera of the patient alone, the risk degree of immune checkpoint storm can be predicted. In the present application, the baseline concentration refers to the concentration of the biomarker in the plasma collected from the patient before medication.

The serological biomarkers for predicting COVID-19 immune checkpoint storm and the application and kit thereof provided in the present application will be illustrated in detail below in conjunction with the following embodiments, which, however, cannot be construed as the limitation to the protection scope of the present application.

### Embodiment 1

Preparation of a liquid chip kit for detecting BTLA, GITR, HVEM, IDO, LAG-3, PD-1, PD-L1, TIM-3, CD28, CD80, 4-1BB, CD27, and CD152 biomarkers.

1. Composition of the Kit
(1) 13-plex-coated microspheres: including encoded microspheres coated with BTLA, GITR, HVEM, IDO, LAG-3, PD-1, PD-L1, TIM-3, CD28, CD80, 4-1BB, CD27, and CD152 capture antibodies, respectively;
(2) 13-plex biotin-labeled detection antibodies: BTLA, GITR, HVEM, IDO, LAG-3, PD-1, PD-L1, TIM-3, CD28, CD80, 4-1BB, CD27, and CD152 detection antibodies labeled respectively with a biotin;
(3) streptavidin-labeled phycoerythrin.

Where, the clone numbers of the BTLA, GITR, HVEM, IDO, LAG-3, PD-1, PD-L1, TIM-3, CD28, CD80, 4-1BB, CD27, and CD152 capture antibodies are 6F7, 110416, LH1, 7H8L17, C9B7W, J116, MIH1, F38-2E2, 10F3, 18, 4B4, O323, 14D3, respectively; the clone numbers of the BTLA, GITR, HVEM, DO, PD-L1, CD28, CD80, and CD152 detection antibodies are MIH26, DT5D3, eBioHVEM-122, 2E2.6, 10F.9G2, 37407, MEM-233, and WKH 203, respectively, and the LAG-3, PD-1, TIM-3, 4-1BB, CD27 detection antibodies are polyclonal antibodies.

2. Preparation Method of the Kit

The following steps are included:
(1) coating corresponding microspheres with corresponding capture antibodies
a. taking carboxyl microspheres, shaking the microsphere suspension by a vortex oscillator for 20 s to mix the microspheres evenly;
b. taking 1.1×10⁶ carboxyl microspheres, transferring them into a centrifuge tube and centrifuging at ≥8000 g for 2 min to precipitate the microspheres;
c. removing the supernatant, adding 100 μL of dH₂O, shaking by a vortex oscillator for 20 s to resuspend the microspheres, centrifuging at ≥8000 g for 2 min to precipitate the carboxyl microspheres; removing the supernatant, adding 80 μL of 100 mmol/L sodium dihydrogen phosphate solution with a pH value of 6.2, and shaking by the vortex oscillator for 20 s to resuspend the washed carboxyl microspheres;
d. adding 10 μL of 50 mg/mL N-hydroxysulfosuccinimide, and shaking by the vortex oscillator gently;
e. adding 10 μL of 50 mg/mL 1-ethyl-3[3-(dimethylamino) propyl] carbodiimide, and shaking by the vortex oscillator gently;
f. incubating at room temperature for 20 min, shaking by the vortex oscillator gently every 10 min, centrifuging at ≥8000 g for 2 min to precipitate the activated carboxyl microspheres;
g. removing the supernatant, adding 50 mmol/L 2-(N-morpholino) ethanesulfonic acid (MES) with a pH value of 5.0, shaking by the vortex oscillator for 20 s to resuspend the activated carboxyl microspheres, centrifuging at ≥8000 g for 2 min to precipitate the washed carboxyl microspheres; repeating this step 2 times, washing 2 times with 50 mmol/L MES with a pH value of 5.0, adding 50 mmol/L MES with a pH value of 5.0, shaking by the vortex oscillator for 20 s, adding 55 μg of BTLA, GITR, HVEM, DO, LAG-3, PD-1, PD-L1, TIM-3, CD28, CD80, 4-1BB, CD27, and CD152 capture antibodies, respectively into the well-mixed microspheres, metering to 500 μL with 50 mmol/L MES with a pH value of 5.0, and mixing evenly by the vortex oscillator; incubating at room temperature on a shaker for 2 h, and centrifuging at ≥8000 g for 2 min to precipitate the coupled microspheres;
h. removing the supernatant, adding 300 μL of PBS-TBN, shaking by the vortex oscillator for 30 s; incubating at room temperature on a shaker for 30 min, and centrifuging at ≥8000 g for 2 min to precipitate the coupled microspheres;
i. removing the supernatant, adding 1 mL of PBS-TBN, shaking by the vortex oscillator for 30 s, centrifuging at ≥8000 g for 2 min to precipitate the coupled microspheres; repeating this step once, and washing twice with PBS-TBN;
j. adding 1 mL of PBS-TBN to resuspend the coupled and washed microspheres, obtaining the conjugates of BTLA, GITR, HVEM, IDO, LAG-3, PD-1, PD-L1, TIM-3, CD28, CD80, 4-1BB, CD27, and CD152 capture antibodies with the microspheres;
k. counting the number of the microspheres with a cell counter, with a concentration of 2.5×10 5 per mL; and storing the coupled microspheres at 4° C. in dark;
(2) biotinylation of corresponding detection antibodies
l. diluting 1 mg of BTLA, GITR, HVEM, IDO, LAG-3, PD-1, PD-L1, TIM-3, CD28, CD80, 4-1BB, CD27, and CD152 detection antibodies respectively with 0.1 mol/L sodium bicarbonate buffer solution with a pH value of 8.0 to 1 mg/mL, with its final volume being 1 mL;
m. fully dialyzing the proteins alternately with 0.1 mol/L sodium bicarbonate buffer solution with a pH value of 8.0;
n. dissolving 1 mg of N-hydroxy succinimide-activated biotin with 1 mL of dimethyl sulfoxide;
o. adding 120 μL of 1 g/L NHSB solution respectively into 1 mL solution of BTLA, GITR, HVEM, IDO, LAG-3, PD-1, PD-L1, TIM-3, CD28, CD80, 4-1BB, CD27, and CD152 detection antibodies; continuously stirring at room temperature and keeping for 2 to 4 h;
p. adding 9.6 μL of 1 mol/L NH₄Cl solution, stirring at room temperature for 10 min, fully dialyzing against PBS at 4° C. to remove the free biotin; screening 1 mL of molecules on the sample over a column, eluting slowly with PBS to collect 1 mL per tube, and eluting the proteins between 1 and 3 mL; the final concentrations of the added samples being 0.5 g/L of sodium azide and 1.0 g/L of BSA; and storing the conjugated products at 4° C. in dark.

### Embodiment 2

Application of BTLA, GITR, HVEM, IDO, LAG-3, PD-1, PD-L1, TIM-3, CD28, CD80, 4-1BB, CD27, and CD152 liquid chip kits in predicting COVID-19 immune checkpoint storm.

1. Experimental Objective

It is to demonstrate that the baseline concentrations of BTLA, GITR, HVEM, IDO, LAG-3, PD-1, PD-L1, TIM-3, CD28, CD80, 4-1BB, CD27, and CD152 at admission are higher in COVID-19 patients with immune checkpoint storm.

2. Experimental Subject
1) Under the premise of informed consent and meeting the inclusion conditions, the enrolled cases were selected and the personal basic information was recorded. The cohorts of COVID-19 patients are 109 COVID patients from Beijing Ditan Hospital, Capital Medical University, including 5 asymptomatic patients, 60 mild or moderate patients, and 44 severe or critically ill patients.
2) The plasma was extracted from the patients at admission before treatment, and the specimens were stored in a refrigerator at −80° C.

3. Preparation of Reagents

The kit prepared in Embodiment 1 was used.
(1) Beads: the desired Beads (microspheres) were ultrasonicated for 30 s, and vortexed for 1 min. Each 60 μL was then taken and added into a Mixing Bottle, and the remaining volume was made up to 3 mL with Bead Diluent. They were mixed thoroughly and stored at 2 to 8° C. for one month.
(2) Quality Control: Controls 1 and 2 (i.e., conventional commercially available recombinant proteins) were respectively dissolved with 250 μL of distilled water, mixed thoroughly by inverting several times, and left for 5 to 10 min. After then, they were respectively transferred into two test tubes and stored at −20° C. for one month.
(3) Standard: The Standard was dissolved with 250 μL of distilled water, mixed thoroughly by inverting several times, and left for 5 to 10 min, then transferred into a test tube which was marked as the Antigen standard vial. Then 7 additional test tubes were taken and marked as S1, S2, S3, S4, S5, S6, and S7, respectively. Into S2, S3, S4, S5, S6, and S7 were respectively added 150 μL of Assay buffer solution. 200 μL of the liquid in the Antigen standard vial was transferred into S1. 50 μL of the liquid in S1 was transferred into S2, and mixed by gently pipetting up-down ten times. 50 μL of the liquid in S2 was transferred into S3, and mixed by gently pipetting up-down ten times. 50 μL of the liquid in S3 was transferred into S4, and mixed by gently pipetting up-down ten times. 50 μL of the liquid in S4 was transferred into S5, and mixed by gently pipetting up-down ten times. 50 μL of the liquid in S5 was transferred into S6, and mixed by gently pipetting up-down ten times. 50 μL of the liquid in S6 was transferred into S7, mixed by gently pipetting up-down ten times, and then stored at −20° C. for one month.
(4) Wash Buffer: 10×WB was placed at room temperature so that the salt therein was fully dissolved. 30 mL WB+270 mL distilled water were formulated to 1×, and stored at 4° C. for one month.
(5) Serum Matrix: 1 ml distilled water was added into SM to fully dissolve it and left for 10 min, then transferred into a test tube and stored at −20° C. for one month.

4. Experimental Procedures:
\textcircled{1} 200 μL of Wash Buffer was added into each well of a 96-well plate which was rinsed by shaking at room temperature for 10 min, and then the Wash Buffer was discarded directly, and the plate was wiped to dry properly.
\textcircled{2} adding 25 μL of:
   Serum Matrix into Background, Standard, and Control;
   Assay Buffer into sample wells;
   Assay Buffer into Background;
   Standard and Control into respective locations;
   samples into corresponding sample wells;
   Beads into each well, respectively, and incubating overnight while shaking at 4° C. in dark.
\textcircled{3} washing twice in a plate-washing machine.
\textcircled{4} adding 25 μL of detection antibodies into each well and shaking at room temperature in dark for 1 h.
\textcircled{5} adding 25 μL of SAPE into each well and shaking at room temperature in dark for 30 min.
\textcircled{6} washing twice in a plate-washing machine, and finally adding 150 μL of sheath fluid to each well for detection on the machine (Luminex system).

5. Experimental Results:

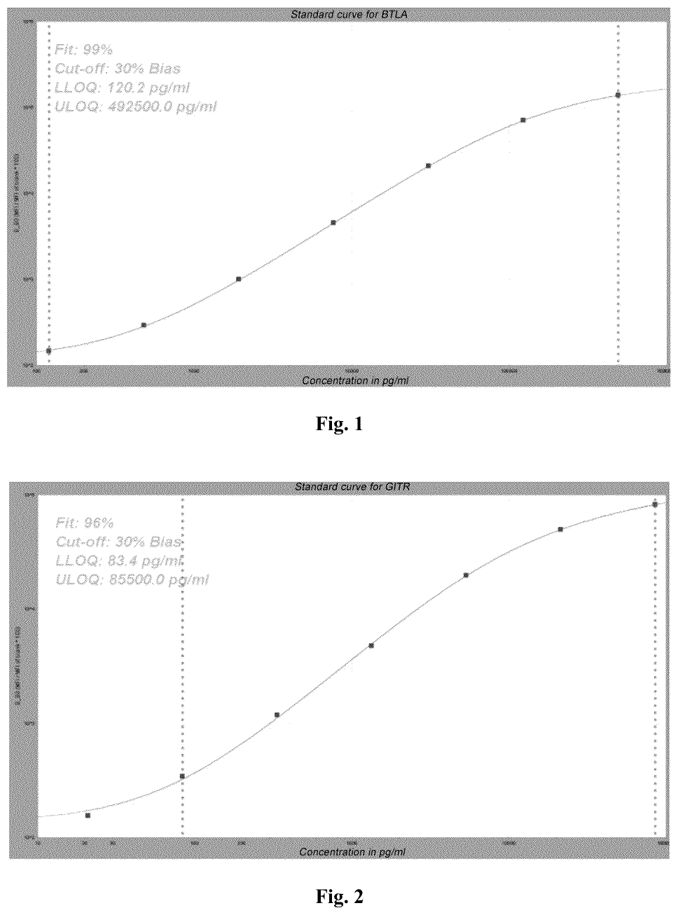

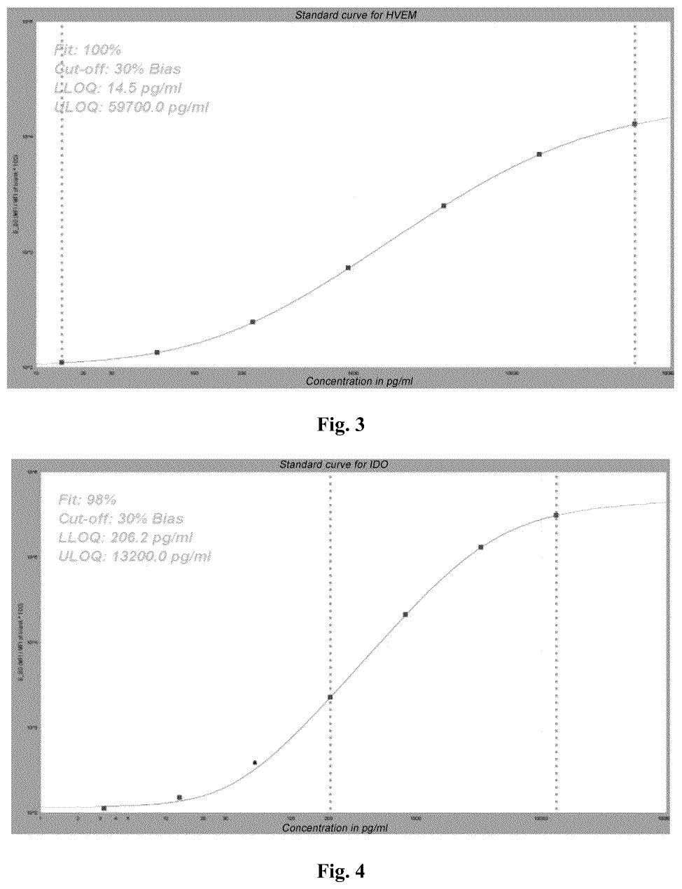

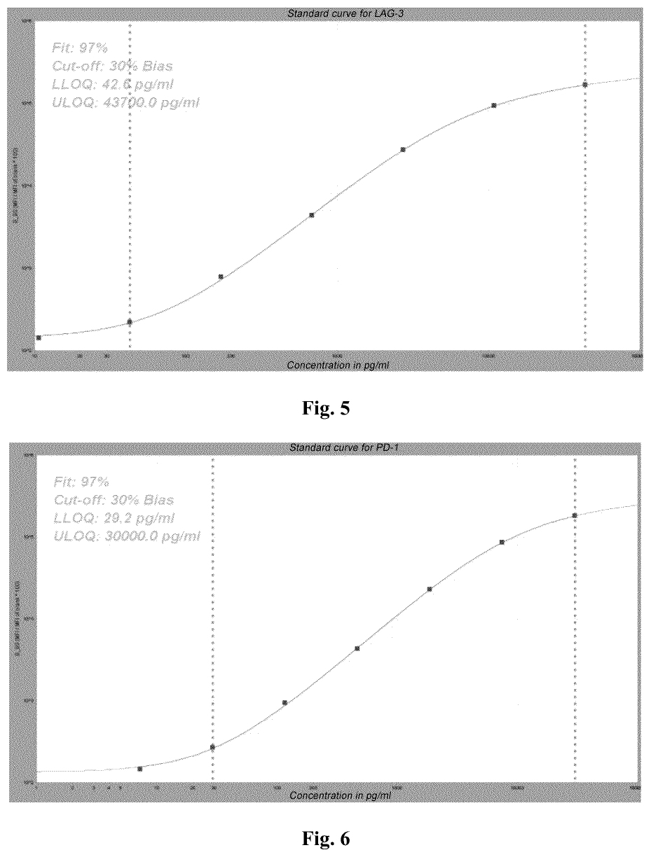

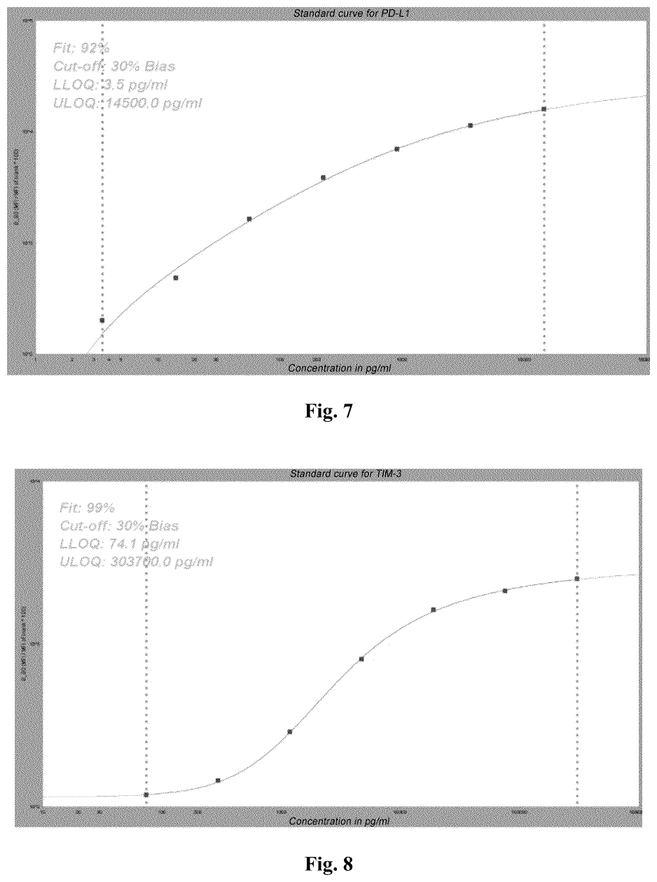

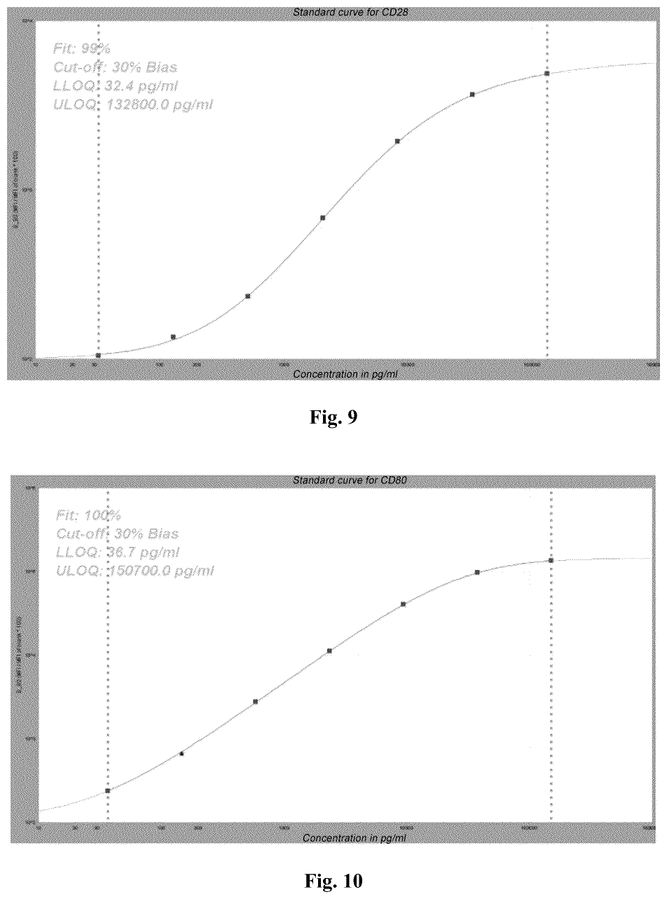

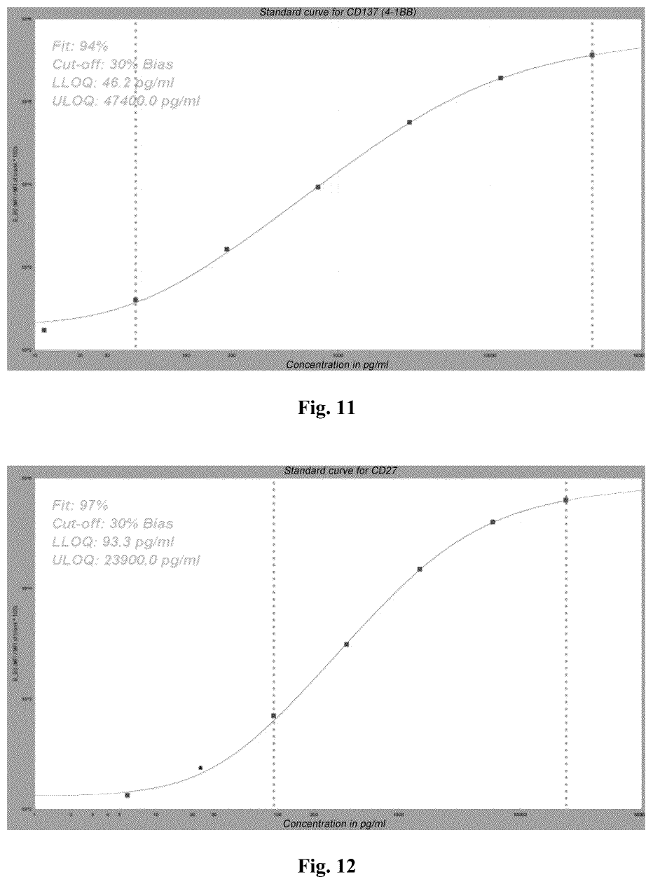

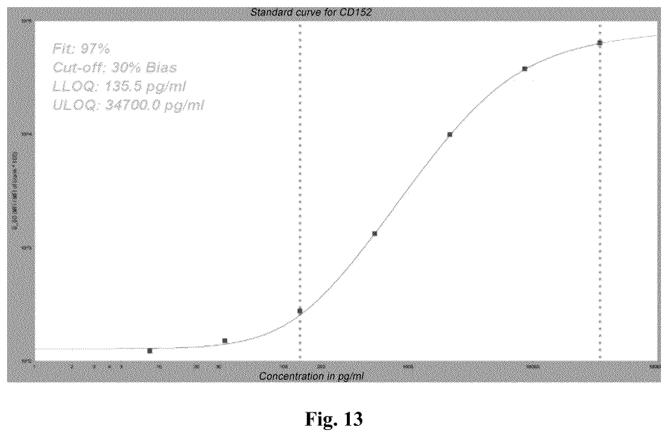

The liquid chip kit was used to detect the baseline concentrations of BTLA, GITR, HVEM, IDO, LAG-3, PD-1, PD-L1, TIM-3, CD28, CD80, 4-1BB, CD27, and CD152 in the sera of three groups of COVID-19 asymptomatic, mild or moderate, and severe or critically ill patients. The concentrations of these 13 serum biomarkers are computed from the fluorescence values read by the machine and the corresponding standard curves, which are shown in FIGS. 1 to 13 and Table 1. The Cut-Off values of the standard curves are 30% Bias (indicating not shown for the moment). In Table 1, Fit indicates Goodness-of-fit, Cut-off: 30% Bias indicates being not shown for the moment; LLOQ indicates the lower limit of quantification; and ULOQ indicates the upper limit of quantification.

TABLE 1
Standard curve parameters of 13 serum biomarkers

| | | Lower Limit of | Upper Limit of |
|---|---|---|---|
| | Goodness-of- | Quantification | Quantification |
| | Fit | (LLOQ)/ | (ULOQ)/ |
| Biomarkers | (Fit) | pg/mL | pg/mL |
| BTLA | 99% | 120.2 | 492500.0 |
| GITR | 96% | 83.4 | 85500.0 |
| HVEM | 100% | 14.5% | 59700.0 |
| IDO | 98% | 206.2 | 13200.0 |
| LAG-3 | 97% | 42.6 | 43700.0 |
| PD-1 | 97% | 29.2 | 30000.0 |
| PD-L1 | 92% | 3.5 | 14500.0 |
| TIM-3 | 99% | 74.1 | 303700.0 |
| CD28 | 99% | 32.4 | 132800.0 |
| CD80 | 100% | 36.7 | 150700.0 |
| 4-1BB | 94% | 46.2 | 47400.0 |
| CD27 | 97% | 93.3 | 23900.0 |
| CD152 | 97% | 135.5 | 34700.0 |

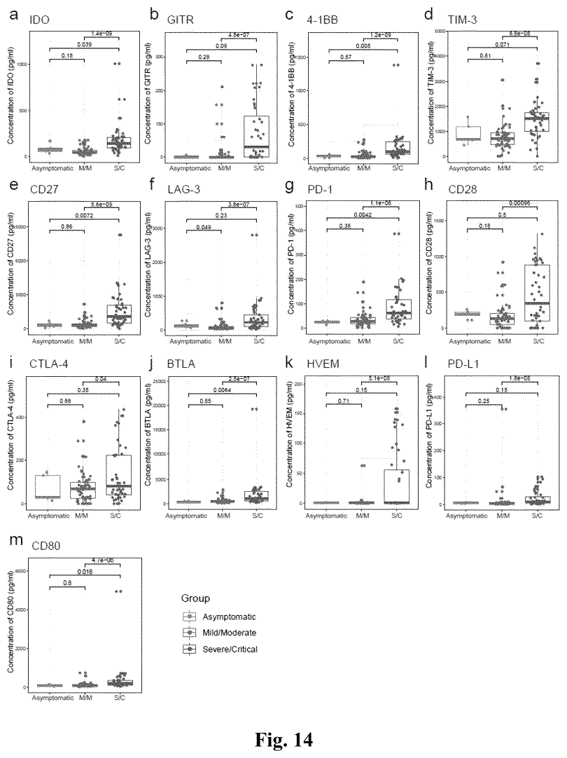

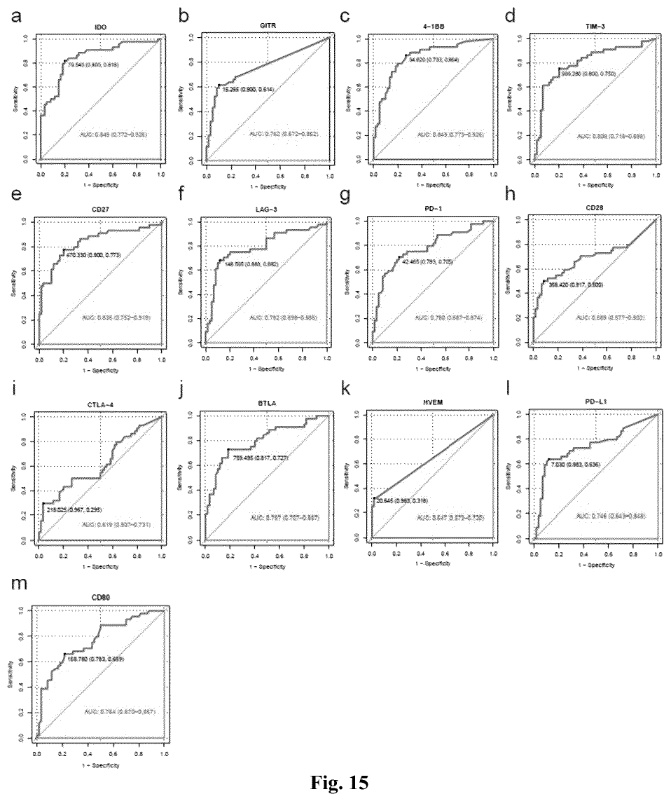

The experimental results show that, for COVID-19 patients, the baseline concentrations of BTLA, GITR, HVEM, IDO, LAG-3, PD-1, PD-L1, TIM-3, CD28, CD80, 4-1BB, CD27, and CD152 in the sera of (severe or critically ill) patients with a high risk of “immune checkpoint storm” are significantly higher than those in patients with a low risk, with the results specifically shown in FIG. 14. The receiver operating characteristic curves (ROC curves) for the prediction of immune checkpoint storm by using the baseline concentrations of BTLA, GITR, HVEM, IDO, LAG-3, PD-1, PD-L1, TIM-3, CD28, CD80, 4-1BB, CD27, and CD152 in the sera of patients were shown in FIG. 15, with the area under the curve (AUC) and 95% confidence interval (95% CI) shown in the figure.

The experimental results also show that, the risk of immune checkpoint storm in COVID-19 patients can be accurately predicted by detecting the baseline concentrations of BTLA, GITR, HVEM, IDO, LAG-3, PD-1, PD-L1, TIM-3, CD28, CD80, 4-1BB, CD27, and CD152 in the sera of the patients at admission, with the prediction accuracy being 79.7%, 76.2%, 64.7%, 84.9%, 79.2%, 78.0%, 74.6%, 80.8%, 68.9%, 76.4%, 84.9%, 83.6%, and 61.9%, respectively.

Finally, it should be noted that the embodiments above are intended only to illustrate the technical solution of the present invention and not to limit. Although the present invention has been described in detail with reference to the embodiments, it should be understood by those of ordinary skill in the art that any modification or equivalent replacement to the technical solution of the present invention does not depart from the spirit and scope of the technical solution of the present invention, which all should be covered by the scope of the claims of the present invention.

What is claimed is:

1. Markers for predicting Coronavirus disease 2019 (COVID-19) immune checkpoint storm, wherein, the markers comprise one or more of BTLA, GITR, HVEM, IDO, LAG-3, PD-1, PD-L1, TIM-3, CD28, CD80, 4-1BB, CD27, and CD152.

2. Application of the markers of claim 1 in the preparation of a kit for predicting COVID-19 immune checkpoint storm.

3. A kit for predicting COVID-19 immune checkpoint storm, wherein, the kit comprises: encoded microspheres coated with one or more capture antibodies of BTLA, GITR, HVEM, IDO, LAG-3, PD-1, PD-L1, TIM-3, CD28, CD80, 4-1BB, CD27, and CD152, respectively, one or more detection antibodies of BTLA, GITR, HVEM, IDO, LAG-3, PD-1, PD-L1, TIM-3, CD28, CD80, 4-1BB, CD27, and CD152 labeled respectively with a biotin, and streptavidin-labeled phycoerythrin.

4. The kit according to claim 3, wherein, the clone numbers of the BTLA, GITR, HVEM, IDO, LAG-3, PD-1, PD-L1, TIM-3, CD28, CD80, 4-1BB, CD27, and CD152 capture antibodies are 6F7, 110416, LH1, 7H8L17, C9B7W, J116, MIH1, F38-2E2, 10F3, 18, 4B4, O323, and 14D3, respectively.

5. The kit according to claim 3, wherein, the clone numbers of the BTLA, GITR, HVEM, IDO, PD-L1, CD28, CD80, and CD152 detection antibodies are MIH26, DT5D3, eBioHVEM-122, 2E2.6, 10F.9G2, 37407, MEM-233, and WKH 203, respectively, and the LAG-3, PD-1, TIM-3, 4-1BB, and CD27 detection antibodies are polyclonal antibodies.

6. The kit according to claim 3, wherein, the encoded microspheres comprise carboxyl microspheres.

7. The kit according to claim 3, wherein, the biotin comprises N-carboxyl succinimide-activated biotin.

8. A method for preparing the kit according to any one of claims 3 to 7, comprising the following steps:
preparation of encoded microspheres coated with capture antibodies: coupling one or more capture antibodies of BTLA, GITR, HVEM, IDO, LAG-3, PD-1, PD-L1, TIM-3, CD28, CD80, 4-1BB, CD27, and CD152 with corresponding encoded microspheres, respectively, to obtain encoded microspheres coated with one or more capture antibodies of BTLA, GITR, HVEM, IDO, LAG-3, PD-1, PD-L1, TIM-3, CD28, CD80, 4-1BB, CD27, and CD152, respectively;
preparation of biotin-labeled detection antibodies: ligating a biotin onto one or more detection antibodies of BTLA, GITR, HVEM, IDO, LAG-3, PD-1, PD-L1, TIM-3, CD28, CD80, 4-1BB, CD27, and CD152, respectively, to obtain one or more detection antibodies of BTLA, GITR, HVEM, IDO, LAG-3, PD-1, PD-L1, TIM-3, CD28, CD80, 4-1BB, CD27, and CD152 respectively labeled with the biotin.
